(19) United States
(12) Patent Application Publication
(10) Pub. No.: US 2021/0030642 A1
(43) Pub. Date: Feb. 4, 2021

(54) TOOTH REMINERALIZATION DENTIFRICE AND METHODS FOR MAKING AND USING THE SAME

(71) Applicant: CAO Group, Inc., West Jordan, UT (US)

(72) Inventor: Steven D Jensen, South Jordan, UT (US)

(21) Appl. No.: 16/917,551

(22) Filed: Jun. 30, 2020

Related U.S. Application Data
(60) Provisional application No. 62/880,403, filed on Jul. 30, 2019.

(51) Int. Cl. A61K 8/19; A61K 8/21; A61Q 11/00

(52) U.S. Cl. CPC A61K 8/19; A61Q 11/00; A61K 8/21

(57) ABSTRACT

A remineralization dentifrice utilizing submicron calcium particles is used to help trigger the oral environment's natural remineralization process. The submicron calcium particles act as a seed to initiate the process. Various sources and forms of calcium are utilized, with or without fluoride ions, to various effect. The dentifrice is delivered as a dry, inorganic powder to the user for maximum effect.

### CROSS-REFERENCES TO RELATED APPLICATIONS

The present application claims priority to prior filed U.S. provisional application No. 62/880,403, filed Jul. 30, 2019, and incorporates the same by reference herein in its entirety.

### FIELD OF THE INVENTION

The present invention relates to the field of dentistry and more particularly relates to a dentifrice and a method of repairing damage to enamel surfaces by harnessing the dental remineralization processes inherent to the oral environment.

### BACKGROUND OF THE INVENTION

Gaps and fissures within teeth are a primary cause of caries because they become a protected haven for food particles and bacteria. Some fissures occur in teeth naturally or might be caused because of trauma, eating habits, natural use, etc. Other fissures or gaps might be caused artificially i.e. during a dental procedure—this is especially the case during restorative procedures. It is well known in the art that most restorations leak between the prosthetic material and tooth. This phenomenon is commonly known in the art as microleakage. It is also known that bonded restorations typically de-bond with respect to time and allow the ingress of bacteria to begin the caries process. Therefore, it is common to find caries underneath old restorations. What is then needed is a procedure in which this microleakage may be stopped, or at least slowed. Filling the gaps and fissures found in teeth, whether caused by natural, accidental, or artificial means, would not only address microleakage and contribute to the longevity of prosthetic materials, but may assist in overall oral care and help reduce the incidents of needing such prosthetic materials in the first place.

The scope of the present invention is to develop materials and methods that utilize the natural remineralization processes of saliva to fill and seal the gaps and fissures commonly found in teeth. Microscopic calcium compounds can infiltrate these fissures and spawn the formation of new mineral growth until such time that the gap becomes filled and eventually sealed as an integral whole.

The present invention is a dentifrice utilizing bioactive calcium compounds which infiltrate micro-fissures in tooth enamel and help trigger and enhance the remineralization of said micro-fissures. The present invention represents a departure from the prior art in that the methods and compounds utilized in the invention represent a proactive step in sealing and healing tooth enamel before greater problems arise.

### SUMMARY OF THE INVENTION

In view of the foregoing disadvantages inherent in the known types of dentifrices, an improved dentifrice and its means of manufacture and use may provide a dentifrice for and a method of restoring initial damage to tooth enamel that meets the following objectives: that the dentifrice be easily manufactured and administered; that materials for the dentifrice be readily and inexpensively sourced; that it be relatively inexpensive to manufacture; and that the method be effective in the treatment of small scale fissures and cracks in enamel surface.

The present invention utilizes calcium compounds that mimic natural materials that actively participate in the remineralization processes of saliva, such as powdered tooth enamel, which is a crystalline derivative of calcium hydroxyl apatite. The present invention also utilizes biologically active materials that undergo chemical replacement while participating in natural re-mineralization such as powdered calcareous seashells, which is a crystalline derivative of calcium carbonate. The present invention utilizes fluoride to modify or convert these various calcium compounds into a more acid resistant state, crystalline calcium fluorapatite, as either a pre-treatment or a post-treatment. The present invention utilizes microscopic calcium compounds preferably submicron particles designed to infiltrate the microscopic gaps and fissures commonly found in teeth.

We define the term: “replacement” using the general geological definition wherein a substance is chemically replaced with another over a rate of time; as an example is the conversion of calcium carbonate to calcium phosphate; wherein an aqueous phosphate is constantly absorbed into a calcium carbonate crystalline matrix such that over a process of time the crystalline calcium carbonate is eventually replaced with crystalline calcium phosphate. Geological examples of replacement are the processes that create petrified wood or fossils for instance. This same process can be utilized in the repair of tooth enamel.

As used in this Specification, the term “submicron” is defined as a substance having a mean particle size distribution below 1 micron (a micrometer, or one millionth of a meter). This would mean that approximately half of all the particles in a powder would have a diameter length less than 1 micron. Powders with mean particle size distributions larger than a micron are also within the scope of this patent, though they are not the preferred embodiment. It is to be understood that smaller particles are better able to infiltrate the microscopic cracks, fissures and gaps found in teeth and, obviously, must be smaller than the cracks they are intended to infiltrate. This Specification may also utilize the term “nanoscale” to refer to sizes on the order of a nanometer (one billionth of a meter) to a micron.

The more important features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also, it is to be understood that the phraseology and terminology employed herein are for description and should not be regarded as limiting.

As such, those skilled in the art will appreciate that the conception, upon which this disclosure is based, may readily be utilized as a basis for the designing of other structures, methods, and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a front elevation of a tooth, in a mouth.

FIG. 2 is a cross-section of the tooth of FIG. 1

FIG. 3 is a close-up view of a microfracture in the tooth of FIG. 2, taken in circle III.

FIG. 4 is a front elevation of a user opening a tube of remineralization dentifrice and applying it to a toothbrush.

FIG. 5 is a front elevation of the user brushing his teeth with the dentifrice.

FIG. 6 is a front elevation of the microfracture of FIG. 2, after brushing, with some microparticles of the dentifrice lodged in the microfracture.

FIG. 7 is a front elevation of the microfracture of FIG. 6, after remineralization has begun.

FIG. 8 is a front elevation of the microfracture of FIG. 7, after remineralization has finished and the microfracture is closed.

### DETAILED DESCRIPTION OF THE PREFERRED EMBODIMENT

With reference now to the drawings, a preferred embodiment of the tooth remineralization dentifrice and the method of its use are herein described. It should be noted that the articles “a”, “an”, and “the”, as used in this specification, include plural referents unless the content clearly dictates otherwise.

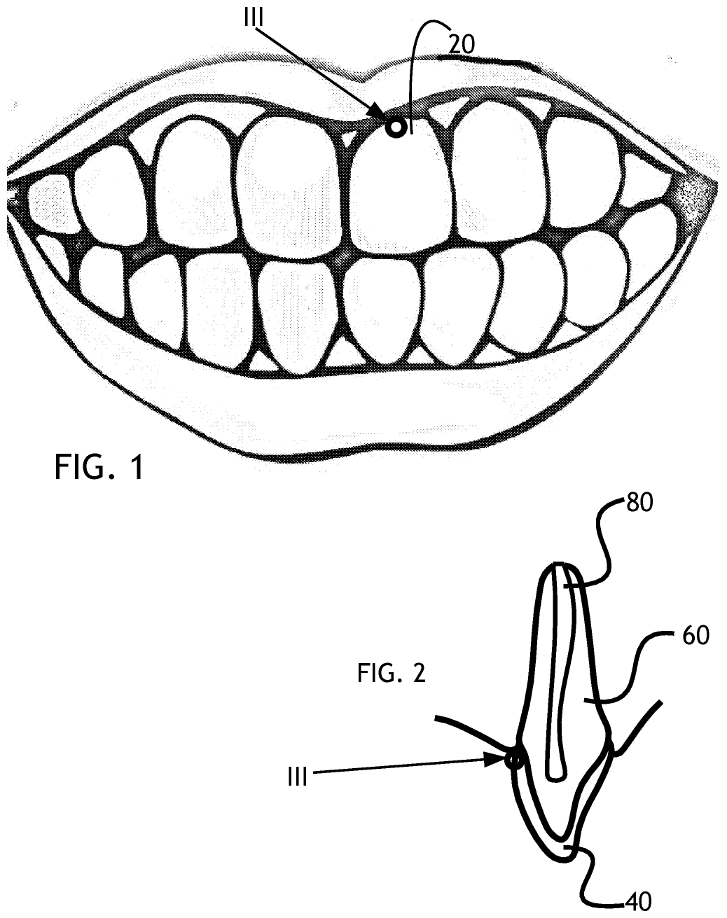

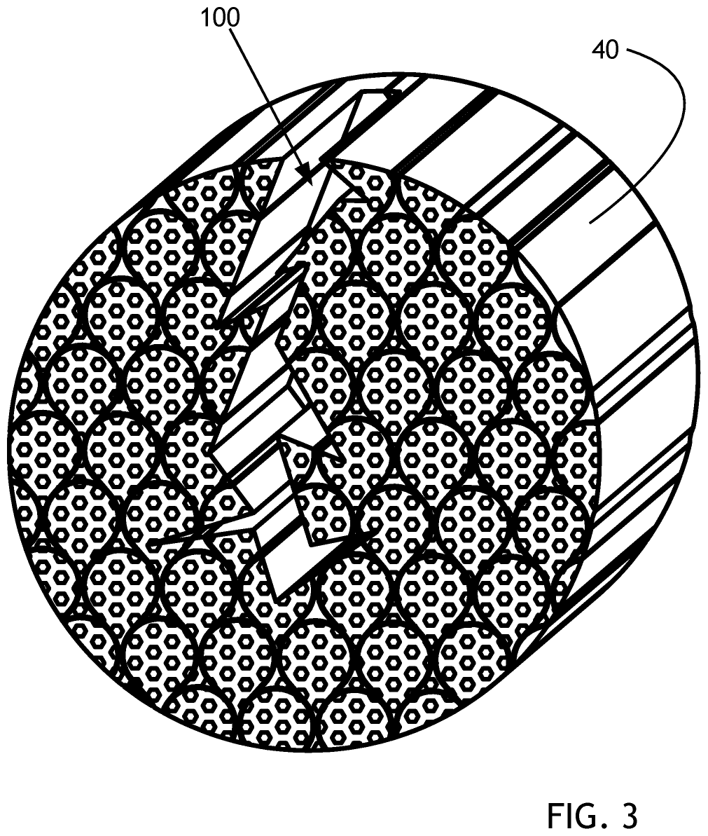

As seen in FIGS. 1 through 3, a tooth 20 has three basic structures: enamel 40, dentin 60, and dental pulp 80, which is essentially the tooth's nerve. The enamel 40 is the hard outer and exposed layer of the tooth 20 on which chewing occurs. The enamel 40 is what will occasionally develop carries (cavities) which slowly progress until the decayed portion reaches the dentin 60 and/or pulp 80. One of the most common methods of a cavity beginning, is to have bacteria invade a microfracture 100 present in the enamel 40. Any given tooth can be assumed to have some microfractures present at any given time due to the daily wear and tear teeth undergo. Microfractures are miniscule and not detectable to the human eye; but, can become a haven for acid producing bacteria which will further damage the enamel 40 and spread the cavity.

The oral environment has a defense against microfractures in the form of the remineralization process. A healthy balance of salts and ions in saliva contributes materials for the enamel 40 to repair these microfractures as they occur. Such remineralization should occur at a faster rate than tooth decay and can in a healthy oral environment.

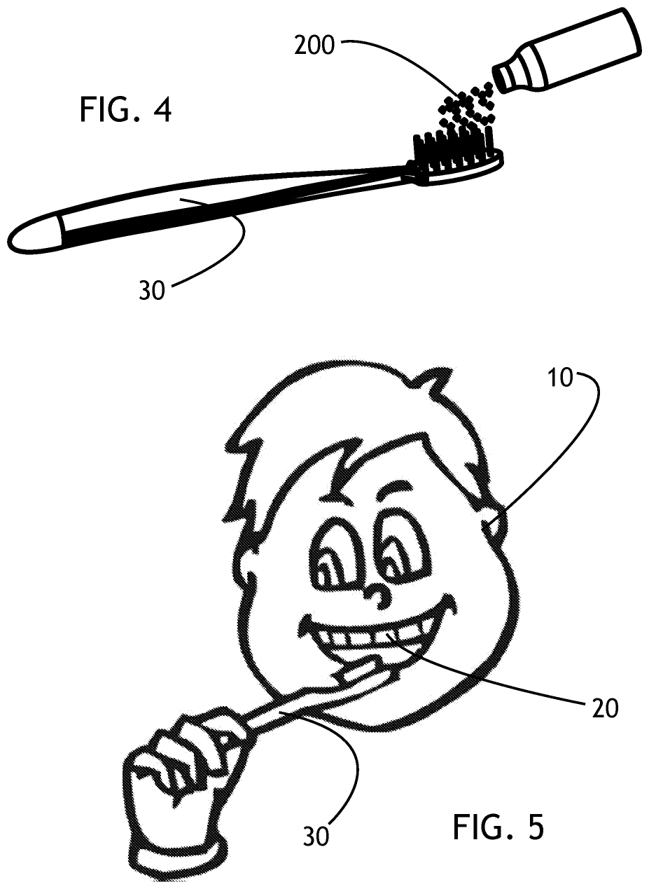

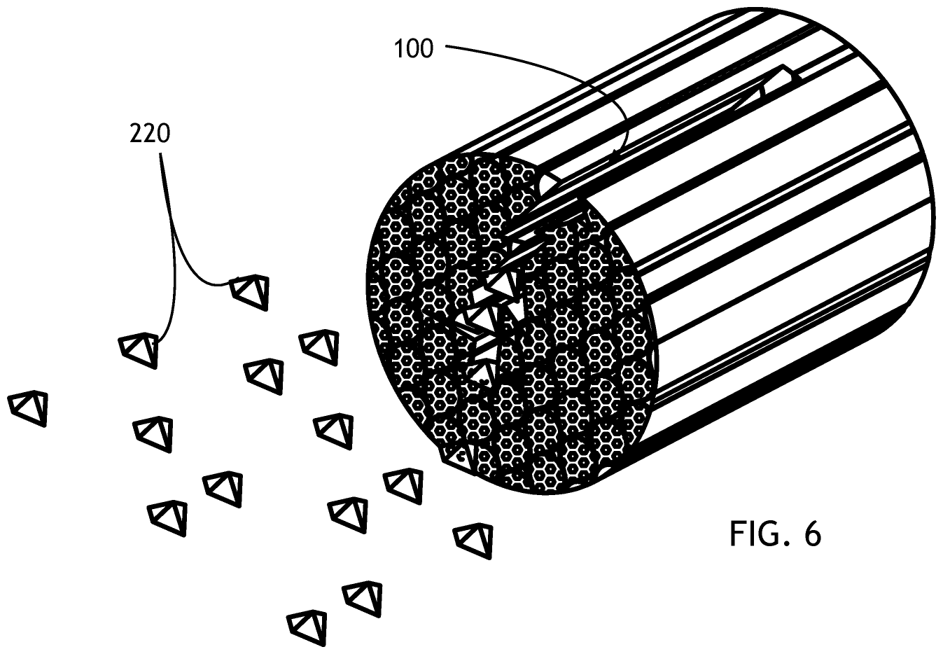

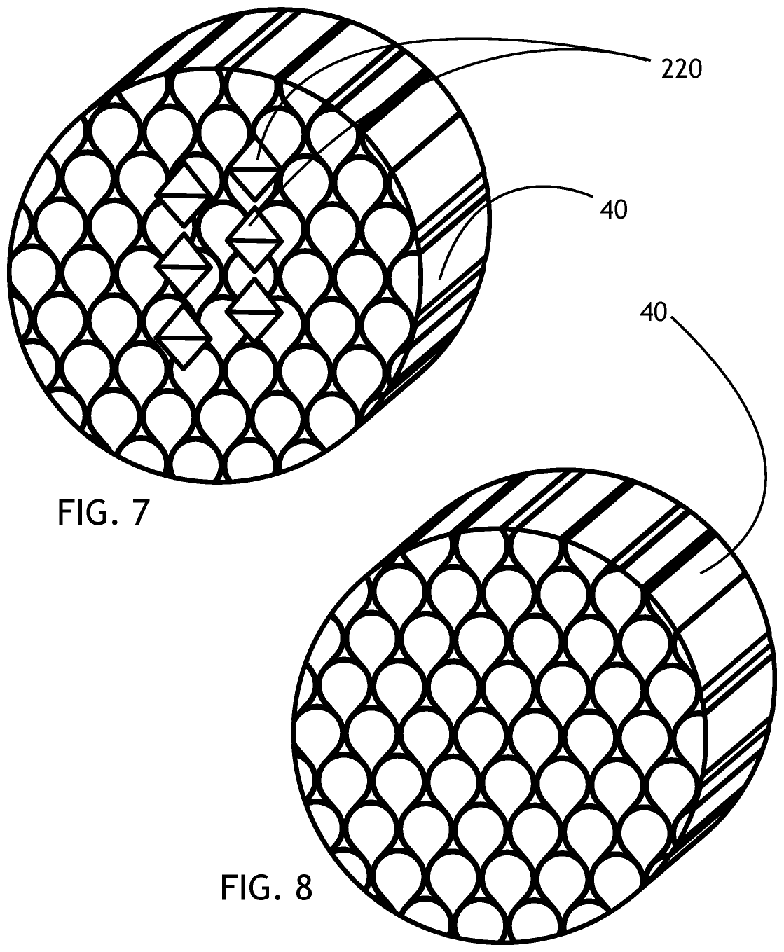

The present invention is a dentifrice which aid the remineralization process and its method of implementation. A dentifrice powder 200 is simply placed on a user's toothbrush 30 (FIG. 4) and the user 10 brushes his or her teeth 20 with the powder 200 (FIG. 5). Granules of the powder 220 lodge in the microfracture 100 (FIG. 6) and act as seeds to encourage remineralization of the microfracture, as shown in FIG. 7. Eventually, the microfracture is sealed and the granules themselves are replaced with healthy enamel structure (FIG. 8).

It is observed that different materials have different spawning characteristics and mineral growth rates in natural and/or artificial saliva; it is within the scope of the invention to utilize all the components found in natural saliva as artificial re-mineralization compounds. A general ranking of the spawning rate and efficiency of tested mineralization compounds with respect to the spawn/growth rate in natural and/or artificial saliva follows:

1. Submicron calcareous seashells (Fastest)

2. Submicron calcium carbonate

3. Submicron enamel

4. Submicron calcium phosphate tribasic (Slowest)

It should be understood that the diversity of seashells is enormous and therefore difficult to classify or isolate groups into a genus with respect to a mineralization rate; what we have found are those seashells that have an amorphous white chalky appearance on the surface have as a generalization the fastest growth rate among seashells. The term “calcareous seashells” is being given to this group due to its specific advantages. Calcareous seashells have a significantly faster spawning/growth rate when introduced into natural and/or artificial saliva. For reasons not fully understood, this substance is even superior to both artificial and natural enamel.

Seashells and/or calcium carbonate have a fast re-mineralization rate as the soluble mineralization salts within saliva are enticed to spawn crystal growth on the surface of these submicron particles. While the new mineral growth is comprised of natural enamel, the submicron particle is still comprised of calcium carbonate. This calcium carbonate core particle is eventually “replaced” using the same saliva remineralization process, which may take years to complete. In this case the remineralization salts of saliva perform two functions:

1. Grow new enamel from a seed crystal of calcium carbonate.

2. Chemically replace the calcium carbonate in a slow process.

It is important to understand that calcium carbonate and phosphates have been utilized in the past in toothpastes; these prior art toothpastes are ineffective at initiating re-mineralization because of 2 factors:

1. Organic compounds such as oils, humectants, emulsifiers, solvents, and carriers (collectively “organic carrier compounds”) that are utilized in formulating the toothpaste coat and saturate the calcium carbonate/phosphate particle and inhibit crystal growth.

2. The mineral particles of the prior art are too large to infiltrate the micro-fissures where they can be lodged sufficiently and permanently remain without being removed by the common forces found in the oral cavity.

Therefore, only a dry dentifrice will trigger remineralization and the particles must be preferentially submicron-sized to infiltrate adequately into the micro-fissures of the teeth to be effective, with further preference being given to particles on a nanoscale.

Introducing fluoride ions into the natural and/or artificial saliva allows the added fluoride to participate in the re-mineralization process and favors the formation of more acid resistant enamel, such as crystalline calcium fluorapatite. Soluble fluoride sources comprise but are not limited to sodium fluoride, potassium fluoride, stannous fluoride, sodium monofluorophosphate, potassium monofluorophosphate and any other soluble fluoride source.

Embodiments of the present invention comprise various components that may be combined in any combination or alone to create a dry dentifrice of the present invention. Examples of such embodiments include:
a. Submicron seashell powder and a soluble fluoride source.
b. Submicron calcium carbonate and a soluble fluoride source.
c. Submicron seashell powder.
d. Submicron calcium carbonate.
e. Submicron enamel powder and a soluble fluoride source.
f. Submicron enamel powder.
g. Submicron calcium phosphate tribasic and a soluble fluoride source.
h. Submicron calcium phosphate tribasic.
i. Submicron seashell powder combined with calcium carbonate.
j. Submicron seashell powder, calcium carbonate, and a soluble fluoride source.
k. Submicron seashell powder combined with powdered enamel.
l. Submicron seashell powder, powdered enamel, and a soluble fluoride source.
m. Submicron seashell powder combined with calcium phosphate tribasic.
n. Submicron seashell powder, calcium phosphate tribasic, and a soluble fluoride source.
o. Submicron seashell powder combined with powdered enamel, calcium carbonate, and calcium phosphate tribasic.
p. Submicron seashell powder, powdered enamel, calcium carbonate, calcium phosphate tribasic, and a soluble fluoride source.

The dentifrices of the present invention may optionally contain sweeteners and flavors to improve the taste in the hope that it will enhance patient brushing compliance. The preferred embodiment may contain flavors and sweeteners in a dry powder state. The dentifrices of the present invention may also optionally contain a surfactant to reduce the surface tension of water. The preferred embodiment may contain surfactants in a dry state. It should be noted that typically surfactants and flavors are organic; however, they will not be organic carrier compounds and will be in a dry state with the remaining dentifrice powder upon application. They will also be in very small quantities, as opposed to the organic carrier compounds which make most of a paste or gel's mass. As such, the surfactants and flavorings will not have had the opportunity to coat the carbonate/phosphate particles before use and, when applied to the oral environment, will still not have an opportunity to do so before the particles have a chance to enter the fissures and/or oral fluids wash the organic compounds away. Therefore, the addition of these organic ingredients will not adversely affect the ability of the particles to penetrate micro-fissures and trigger remineralization.

An embodiment of the present invention loads dry dentifrice compositions into wax paper or plastic tubes that can be torn open when ready to use and the contents poured out onto a toothbrush in a dry, powdered state. Further embodiments contemplate a method of loading a dry dentifrice composition into a plastic, paper, or wax paper tube and sealing the ends. Thereafter the tube is unsealed or simply torn open when ready to use and the contents poured out onto a toothbrush.

An embodiment of the present invention comprises a method of brushing one's teeth with a dry composition of the present invention so that a small portion of the particles infiltrate the microscopic gaps and fissures of the teeth and become lodged therein. Thereafter these particles actively participate in the natural and/or artificial re-mineralization processes of saliva.

Although the present invention has been described with reference to preferred embodiments, numerous modifications and variations can be made and still the result will come within the scope of the invention. No limitation with respect to the specific embodiments disclosed herein is intended or should be inferred.

What is claimed is:

1. A method of promoting enamel remineralization, the method comprising:
providing a powdered dentifrice having an inorganic component with a submicron mean particle distribution;
applying said dentifrice to a surface;
allowing at least one inorganic particle in the dentifrice to lodge in a fracture in said surface;
allowing said microfracture to remineralize around the at least one inorganic particle;
wherein the presence of the at least one inorganic particle will encourage the remineralization process and the dentifrice does not have organic carrier compounds that coat the particles and inhibit crystal growth.

2. The method of claim 1, the inorganic component of the powdered dentifrice being comprised of at least one calcium compound.

3. The method of claim 2, the at least one calcium compound being selected from the set of calcium compounds consisting of: calcareous seashells, calcium carbonate, powdered enamel, and calcium phosphate tribasic.

4. The method of claim 3, the powdered dentifrice further comprising a soluble fluoride source.

5. The method of claim 2, the powdered dentifrice further comprising a soluble fluoride source.

6. The method of claim 1, the powdered dentifrice further comprising a soluble fluoride source.

7. A dentifrice to promote enamel remineralization, the dentifrice comprising a
submicron dry powder containing at least one calcium compound and no organic carrier compounds.

8. The dentifrice of claim 7, the at least one calcium compound being selected from the set of calcium compounds consisting of: calcareous seashells, calcium carbonate, powdered enamel, and calcium phosphate tribasic.

9. The dentifrice of claim 8, further comprising a soluble fluoride source.

10. The dentifrice of claim 7, further comprising a soluble fluoride source.
